(19) United States
(12) Patent Application Publication
(10) Pub. No.: US 2005/0096560 A1
(43) Pub. Date: May 5, 2005

(54) Reusable airflow sensor

(75) Inventors: Susan S. Alfini, Champlin, MN (US); William H. Ham, Mound, MN (US)

Correspondence Address: NIKOLAI & MERSEREAU, P.A., 900 SECOND AVENUE SOUTH, SUITE 820, MINNEAPOLIS, MN, 55402, US

(73) Assignee: Dymedix, Corp., Minneapolis, MN (US)

(21) Appl. No.: 10/702,623

(22) Filed: Nov. 4, 2003

(51) Int. Cl. A61B 5/08; B29C 39/00

(52) U.S. Cl. 600/538; 600/529; 264/239

(57) ABSTRACT

A reusable airflow sensor for monitoring respiratory activity of patients in a sleep lab setting comprises a polyvinylidne fluoride (PVDF) film totally encased within a thermally conductive plastic material that is non-hydroscopic and therefore impervious to moisture which allows resterilization without comprising the electrical performance of the sensor.

### BACKGROUND OF THE INVENTION

I. Field of the Invention

The present invention relates generally to airflow sensors, and more particularly to airflow sensors for use in monitoring inspiratory and expiratory respiratory activity of patients undergoing observation in a sleep laboratory setting.

II. Discussion of the Prior Art

In the following U.S. Patents to Peter Stasz et al, there are described several improvements in airflow sensors for use in sleep studies on patients:

U.S. Pat. Nos.
5,311,875;
6,254,545;
6,485,432;
6,491,642; and
6,551,256.

The teachings of all of the listed patents are incorporated by reference into this patent application.

Each of the above-mentioned patents describes an airflow sensor that takes advantage of the piezoelectric and thermoelectric properties of polyvinylidene fluoride (PVDF) films to provide an electrical signal proportional to temperature changes, such as may be caused by inspiratory and expiratory airflow and to sound vibrations resulting from snoring episodes. The sensors described in each of the foregoing patents are formed as a laminated structure in which the PVDF film is sandwiched between outer and inner layers of foam and woven material that are adhesively bonded to one another and to the PVDF film layer. Also, in each of the prior art arrangements, the sensor assembly is provided with an adhesive layer on an exposed outer surface thereof, allowing the sensor to be affixed to a subject's skin, e.g., such as on the upper lip or another area where vibrations due to snoring are present.

Given the fabrication approach described in the Stasz et al patents identified above, the sensors resulting therefrom are only suitable for a single use. The laminated tape and film structure tends not to be moisture impervious, such that sterilization allows ingress of sterilant between the layers in the lamination creating a short circuit between the metalization layers. Moreover, the adhesive employed to secure the sensor to the patient is compromised upon the removal of the sensor, precluding subsequent reattachment.

A need therefore exists for a method of fabricating an airflow sensor that is reusable with a given patient or with a plurality of different patients. To achieve this result, the airflow sensor must be repeatedly sterilizable and designed to be held in place at a desired site on the patient by means other than adhesive applied to the device at the time of manufacture. The present invention provides a solution to the aforementioned drawbacks of the prior art.

### SUMMARY OF THE INVENTION

The present invention is directed to a method of making a reusable airflow sensor and the resulting product. The method involves the steps of providing a mold with a mold cavity of a predetermined shape configuration and also providing a PVDF film of a size to fit within the cavity of the mold. The film includes a conductive material adhered to opposed major surfaces thereof. Conductive leads are affixed to the conductive material on the opposed major surfaces of the film and then the assembly is inserted into the mold cavity with the leads extending beyond the cavity. A thermally conductive, moisture impervious plastic is then injected into the mold to fill the cavity and to totally encapsulate said film. Once the plastic has solidified, the device is removed from the mold. The plastic employed is moisture impervious and since the PVDF film is totally encapsulated, moisture is precluded from entering the device and creating a short circuit between the metalization layers. The mold cavity is designed so that the overall thickness of the airflow sensor allows the sensor to be flexed to accommodate the contour of the skin surface on which the sensor is mounted.

### BRIEF DESCRIPTION OF THE DRAWINGS

The foregoing features, objects and advantages of the invention will become apparent to those skilled in the art from the following detailed description of a preferred embodiment, especially when considered in conjunction with the accompanying drawings in which:

FIG. 1 is a perspective view of the airflow sensor fabricated in accordance with the teachings of the present invention;

FIG. 2 is an exploded cross-sectional view taken along the line 3-3 in FIG. 2;

FIG. 3 is a partial perspective view of a mold in which the airflow sensor of the present invention is formed; and

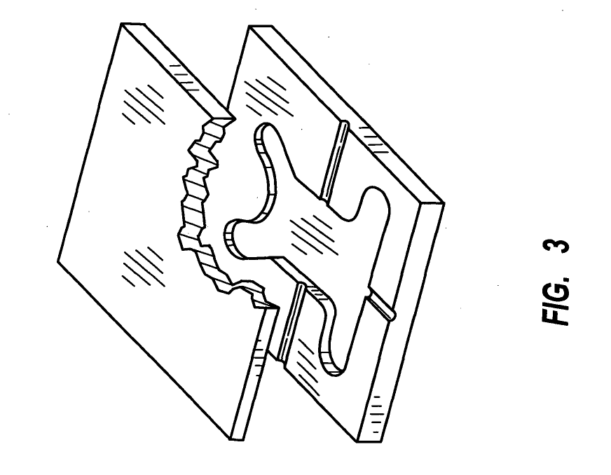

FIG. 4 is a front view of the sensor of the present invention as it is applied to a patient during use.

### DESCRIPTION OF THE PREFERRED EMBODIMENT

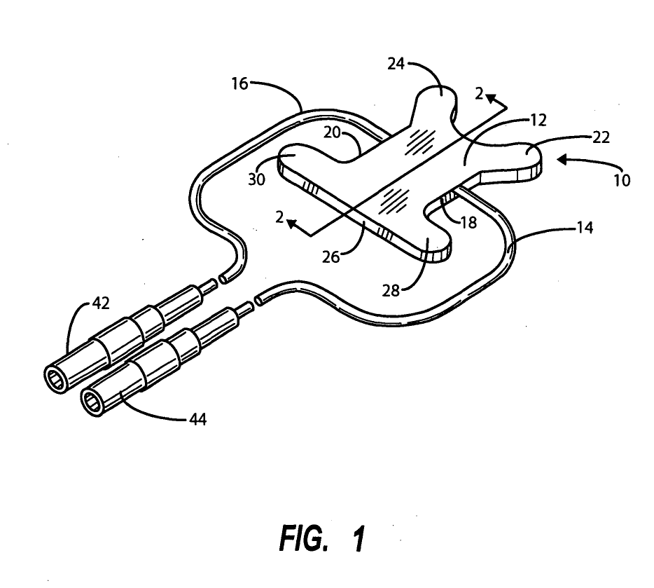

Referring first to FIG. 1, there is illustrated an airflow sensor fabricated using the method of the present invention. The sensor is identified generally by 10 and is seen to include a molded plastic body 12 of a predetermined shape configuration and having first and second electrical leads 14 and 16 extending out from opposed side edges 18 and 20 of the sensor body 12.

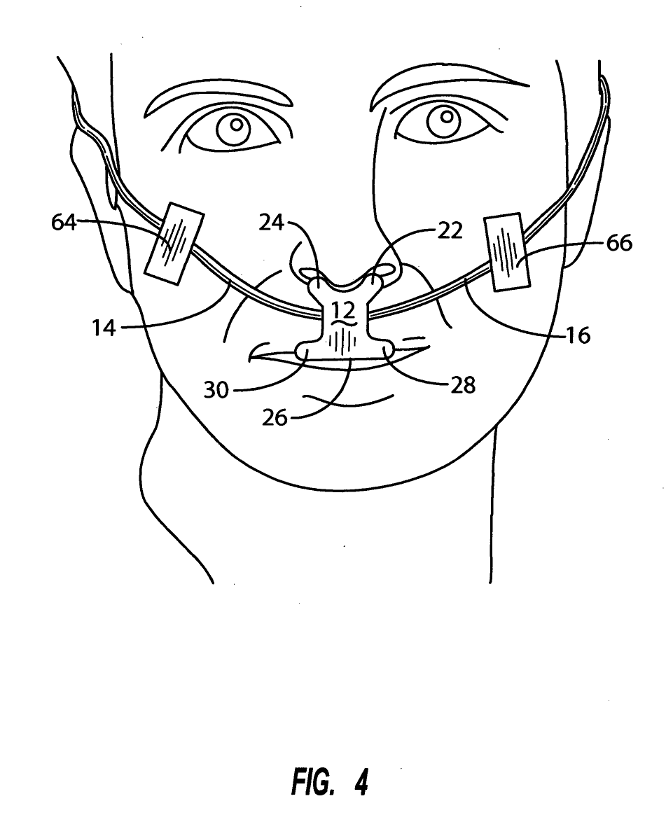

While the sensor body 12 may take on a variety of shape configurations, when the airflow sensor is designed for placement on a subject's upper lip as shown in FIG. 4, the sensor preferably has a pair of arcuate lobes 22 and 24 projecting obliquely from a top edge thereof and spaced so that they will be aligned with the subject's nasal openings. Projecting laterally from the side edges of the body approximate the lower edge 26 are lobes 28 and 30. As seen in FIG. 4, a portion of the sensor body including the lobes 28 and 30 extend over the edge of the subject's upper lip and in front of the subject's mouth.

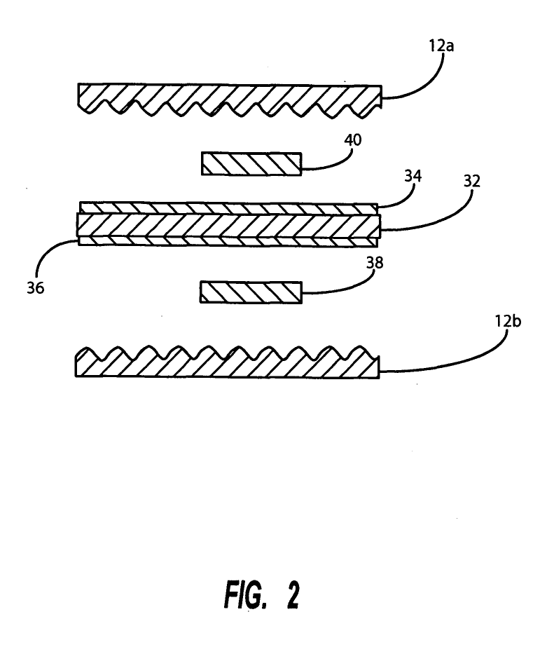

The exploded cross-sectional view of FIG. 2 will aid the reader in understanding the internal construction of the airflow sensor 10. In this drawing, the body 10 is shown as being split along a mid-line into two parts 12a and 12b in order to expose the internal structures comprising the airflow sensor. Here it can be seen that there is encapsulated within the plastic body PVDF film 32 whose shape in a plan view corresponds to the shape of the body member 12 shown in FIG. 1. Electrodeposited or otherwise formed on the opposed major surfaces of the film 32 are metallization layers 34 and 36. Conductively secured to the metallization layers are metal tabs, as at 38 and 40, which are joined to electrical conductors in the elongated leads 14 and 16. Affixed to the proximal end of the leads are connectors 42 and 44 that are adapted to mate with input terminals of a signal processing module, which may be of the type described in the Stasz, et al., patent application Ser. No. 09/860,089, filed May 17, 2001, entitled “SIGNAL PROCESSING CIRCUIT FOR PYRO/PIEZO TRANSDUCER”.

In order to encapsulated the PVDF film, the metallization layers thereon and the distal end portions of the leads including the metal tabs 38 and 40 there is provided a mold, indicated generally by 50, and comprising a mold base 52 in which is formed a mold cavity 54. The shape of the mold cavity, of course, defines the shape of the body 12 of the air sensor. The mold cavity preferably has a height dimension in a range from about 0.24 mm to about 5 mm. Channels as at 56 and 58 are formed in the mold base to accommodate the leads allowing them to extend outward from the mold base 52. A further channel 60 is formed in the mold base and comprises an injection port through which a thermal plastic can be injected. Once the film with its leads attached are disposed in the mold cavity 54, a mold top 62 is clamped to the mold base and subsequently the molten plastic is injected through the injection channel 60 to thereby fill the mold cavity and totally encapsulate the PVDF film 32 and the distal end portions of the leads including tabs 38 and 40. The plastic is allowed to solidify and the finished device is then removed from the mold.

The plastic employed should be moisture impervious, i.e., non-hydroscopic, and preferably should exhibit a resistivity of at least 10¹⁰ ohm-cm. The plastic should also exhibit high thermal conductivity. Suitable candidates for the plastic employed in encapsulating the pyro/piezo film include, but are not necessarily limited to polyethylene, polystyrene, acrylonitrile butadiene styrene, polyphenylene sulfite, silicone vulcanite, and polyamide 4,6. However, by appropriate choice of plastic body thickness, other plastics may also be used. For example, a relatively thin layer of polyvinyl chloride exhibits sufficient thermal conductivity to create a measurable electrical response. Thus, there is no need to limit the choice of plastics employed to a class of thermally conductive plastics although it must pass biocompatibility standards. However, the thickness of the sensor body may be increased when plastic employed exhibits a thermal conductivity of 1.0 W/mK or greater.

As is illustrated in FIG. 4, the reusable airflow sensor of the present invention can be positioned on the upper lip of the subject with the lobes 22 and 24 aligned with the subject's nostrils and with the lower edge 26 extending below the lip in front of the mouth. The device can be held in place by adhesive tabs 64 and 66 that affix the leads 14 and 16 to the subject's cheeks. Because of the temperature/vibration sensing PVDF film 32 and the metallization layers thereon are encapsulated by the plastic, the sensor can be removed following use and subjected to sterilizing fluids without introduction of moisture into the interior. As such, the device of the present invention is completely reusable.

The system of the present invention has many applications and can be configured in a number of ways. The foregoing description is provided to comply with the disclosure requirements of the patent laws, but is not intended to be limiting. The scope of the invention is, of course, defined exclusively by the following claims.

1. A method of making a reusable airflow sensor comprising the steps of:
(a) providing a mold with a mold cavity of a predetermined shape configuration;
(b) providing a polyvinylidene fluoride film of a size to fit within said cavity, said film including a conductive material adhered to opposed major surfaces of said film;
(c) affixing conductive leads to the conductive material on the opposed major surfaces;
(d) inserting said film into said cavity with said leads extending beyond the cavity;
(e) injecting a non-hydroscopic plastic into said mold to fill the cavity and encapsulate said film; and
(f) removing the product of step (e) from the mold following solidification of the plastic.

2. The method of claim 1 wherein the plastic is thermally conductive.

3. The method of claim 1 wherein the plastic is such that the product of step (f) is repeatedly sterilizable.

4. The method of either claim 1 or claim 2 wherein the plastic exhibits a resistivity of at least 10¹⁰ ohm-cm.

5. The method as in claim 1 wherein a height dimension of the mold cavity is in a range from about 0.24 mm to 5 mm.

6. A reusable airflow sensor comprising:
(a) a film of polyvinylidene fluoride material having opposed major surfaces, the opposed major surfaces having a layer of metalization thereon with a pair of elongated conductive leads, each having one end thereof individually affixed to the metalization layers on the opposed major surfaces; and
(b) a sterilizable, medical-grade, non-hydroscopic plastic totally encapsulating said film, metalization layers and said at least one ends of said pair of leads.

7. The reusable airflow sensor as in claim 6 wherein said film, said layers of metalization and said at least one ends of said pair of leads are hermetically sealed within said plastic.

8. The reusable airflow sensor as in either claim 6 or claim 7 wherein the plastic is selected from a group consisting of polyamide 4,6, polyvinyl chloride, polyethylene, polystyrene, thermally conductive acrylonitrile butadiene styrene, thermally conductive polyphenylene sulfide, and thermally conductive silicone vulcanite.

9. The reusable airflow sensor of claim 7 wherein the plastic encapsulating said film, said layers of metalization and said at least one ends allows the airflow sensor to be flexible.

10. The reusable airflow sensor as in claim 6 wherein the plastic has a high resistivity in a range from about 10¹⁰ to 10¹⁵ ohm-cm.

11. The reusable airflow sensor as in claim 6 wherein the plastic is selected from the group consisting of acrylonitrile butadiene styrene, polyphenylene sulfide, silicone vulcanite and polyamide 4,6.

12. The reusable airflow sensor as in claim 6 wherein the thermal conductivity of said plastic is in a range from about 1.0 W/mk to 20 W/mk.
